(19) United States
(12) Patent Application Publication
(10) Pub. No.: US 2014/0255480 A1
(43) Pub. Date: Sep. 11, 2014

(54) PHARMACEUTICAL FORMULATION OF NANONIZED FENOFIBRATE

(71) Applicants: Catherine HERRY, FRANCE (FR); Pascal OURY, Le Chesnay (FR)

(72) Inventors: Catherine HERRY, FRANCE (FR); Pascal OURY, Le Chesnay (FR)

(73) Assignee: ETHYPHARM, Saint-Cloud (FR)

(21) Appl. No.: 14/246,751

(22) Filed: Apr. 7, 2014

Related U.S. Application Data
(63) Continuation of application No. 13/141,197, filed on Sep. 7, 2011, now abandoned.

(51) Int. Cl. A61K 31/216; A61K 9/20; A61K 9/14; A61K 9/50; A61K 31/192; A61K 9/16

(52) U.S. Cl. CPC A61K 31/216; A61K 31/192; A61K 9/1676; A61K 9/146; A61K 9/145; A61K 9/5078; A61K 9/2086
USPC 424/452; 514/543; 514/571; 424/489; 424/465; 424/494; 427/2.14

(57) ABSTRACT

The invention relates to a pharmaceutical formulation of nanonised fenofibrate, to a method for preparing same, and to the uses thereof.

The present invention relates to a novel pharmaceutical composition containing fenofibrate. Fenofibrate, also known as 1-methylethyl 2-[4-(4-chlorobenzoyl)phenoxy]-2-methyl-propanoate, is a lipid-regulating agent. It has a molecular weight of 360.83 g/mol and a melting point in the crystalline state between 79° C. and 82° C.

Fenofibrate is recommended in the treatment of adult endogenous hyperlipidemia, hypercholesterolemia and hypertriglyceridemia. A treatment of 300 mg to 400 mg of fenofibrate per day enables a 20% to 25% reduction of cholesterolemia and a 40% to 50% reduction of triglyceridemia to be obtained.

The major fenofibrate metabolite in the plasma is fenofibric acid, which is the active form of fenofibrate. The melting point of fenofibric acid in the crystalline state is between 179° C. and 183° C. The plasma elimination half-life of fenofibric acid is roughly 20 hours. Its maximum plasma concentration is reached, on average, five hours after ingestion of the drug. The mean plasma concentration is roughly 15 μg/ml for a dose of 300 mg fenofibrate per day. This level is stable throughout the treatment.

Fenofibrate is a very slightly water-soluble active ingredient whose absorption in the digestive tract is limited. An increase in its solubility or its rate of solubilization results in better digestive absorption.

Because of this low affinity for water and its hydrophobic nature, fenofibrate is absorbed better after the ingestion of food compared to under conditions of fasting. This phenomenon is called the food effect. It is particularly important when the absorption of fenofibrate taken with a fatty meal is compared to the absorption of fenofibrate under conditions of fasting.

The principal disadvantage of the food effect is that the patient's diet must be monitored during the treatment. Moreover, as fenofibrate is absorbed better with a fatty meal, it is usually taken after a fatty meal, which is incompatible with a treatment for hyperlipidemia, hypercholesterolemia or hypertriglyceridemia.

Various approaches have been explored in order to increase the rate of solubilization of fenofibrate: micronization of the active ingredient, addition of a surfactant, comicronization of fenofibrate with a surfactant and submicronization of fenofibrate.

Patent EP256933 describes fenofibrate granules in which the fenofibrate is micronized in order to increase its bioavailability. The crystalline fenofibrate microparticles are less than 50 μm in size. The binder used is polyvinylpyrrolidone. The document suggests other types of binder, such as methacrylic polymers, cellulose derivatives and polyethylene glycols. The granules described in the examples of EP256933 are obtained by a method using organic solvents.

Patent EP330532 proposes improving the bioavailability of fenofibrate by comicronizing it with a surfactant, such as sodium lauryl sulfate. The comicronizate is then granulated by wet granulation in order to improve the flow capacities of the powder and to facilitate the transformation into gelatin capsules. This comicronization enables a significant increase in bioavailability compared to the use of fenofibrate described in EP256933. The granules described in EP330532 contain polyvinylpyrrolidone as a binder.

This patent teaches that the comicronization of fenofibrate with a solid surfactant significantly improves the bioavailability of the fenofibrate compared to the use of a surfactant, of micronization or of the combination of a surfactant and of micronized fenofibrate.

Patent WO98/31361 proposes improving the bioavailability of the fenofibrate by attaching to a hydrodispersible inert support micronized fenofibrate, a hydrophilic polymer and, optionally, a surfactant. The hydrophilic polymer, identified as polyvinylpyrrolidone, represents at least 20% by weight of the composition described above.

This method makes it possible to increase the rate of dissolution of the fenofibrate, and also its bioavailability. However, the preparation method of that patent is not entirely satisfactory since it requires the use of a considerable amount of PVP and of the other excipients. The example presented in that patent application refers to a composition containing only 17.7% of fenofibrate expressed as a mass ratio. This low mass ratio for fenofibrate leads to a final form which is very large in size, hence a difficulty in administering the desired dose of fenofibrate, or the administration of two tablets.

Application WO2004/041250 relates to fibrate compositions comprising fibrate particles of submicron average size smaller than 2000 nm.

The nanonization of fenofibrate makes it possible to further improve its dissolution profile. However, several disadvantages result from this decrease in size. The nanonized particles are not stable and tend to agglomerate. In addition, grinding at roughly nanometer dimensions has a high risk of changing the crystalline form of the active ingredient from a polymorphic form to an amorphous form. However, the amorphous form is less stable than the crystalline form and will evolve into the crystalline form over time with a potential impact on the dissolution behavior in humans.

It was discovered in the context of the present invention that the conditions of wet milling enabling the production of nanonized fenofibrate can be optimized in order to maintain the fenofibrate in crystalline form during grinding and to obtain greater bioavailability and stability while preserving the absence of the food effect.

The present invention thus relates to an aqueous suspension containing fenofibrate and/or fenofibric acid in suspension, a cellulose derivative as a solubilization adjuvant and a surfactant, wherein the fenofibrate and/or the fenofibric acid are in crystalline form with melting points in differential scanning calorimetry (DSC) of 79° C. to 82° C. and 179° C. to 183° C., respectively, and the average particle size (D50) of the fenofibrate and/or the fenofibric acid is less than 250 nm, preferably 180 nm. Differential scanning calorimetry is used to characterize the melting or crystallization points of crystalline solids, or the vitreous transition temperatures of amorphous solids.

Preferably, the aqueous suspension of the invention comprises fenofibrate or fenofibric acid.

Preferably, 90% of the particles (D90) have a size less than 500 nm, preferably 400 nm. The size of the particles is determined by laser diffraction or light scattering particle analysis.

Advantageously, the suspension of the invention further comprises a phospholipid, preferentially soy lecithin.

The addition of a phospholipid to the suspension improves the stability of the suspension. Over time, the suspension sediments. In the absence of phospholipid, its suspension after agitation is difficult or even impossible: the solid phase of the suspension forms a cake that is difficult to disperse. When a phospholipid is added to the suspension, simple agitation makes it possible to suspend the solid phase. The phospholipid also has the advantage of enabling the formation of micelles in water, promoting the availability of the fenofibrate for absorption.

Advantageously, the proportion of phospholipid in the suspension of the invention is between 0.25% and 10%, preferably between 0.3% and 5%, more preferably between 0.5% and 1%, by weight of the suspension.

Advantageously, the total proportion of the fenofibrate and the fenofibric acid in the suspension of the invention is between 10% and 20%, preferably between 12% and 17%, by weight of the suspension.

The binding cellulose derivative represents between 0.1% and 10%, preferably between 1% and 5%, by weight of the composition.

Advantageously, the cellulose derivative is hydroxypropyl methylcellulose (HPMC).

Hydroxypropyl methylcellulose is preferably chosen, the apparent viscosity of which is between 3 mPa·s (cP) and 15 mPa·s (cP), and even more preferably between 3 mPa·s (cP) and 6 mPa·s (cP), such as for example Pharmacoat 603®.

The surfactant is selected from surfactants that are solid or liquid at room temperature, for example sodium lauryl sulfate, Polysorbate 80 or Montane 20, preferably sodium lauryl sulfate.

The ratio of the fenofibrate and/or the fenofibric acid to the HPMC is preferably between 5:1 and 15:1.

The surfactant represents between 0.1% and 3%, preferably between 0.3% and 1%, by weight relative to the weight of the suspension.

The suspension of the invention may also contain at least one excipient such as antifoaming agents, for instance dimethicone and simethicone.

The present invention also relates to a method for preparing the suspension of the invention, comprising the following steps:
(a) mixing part of the proportion of cellulose derivative in the suspension, the surfactant and, optionally, the phospholipid with water under agitation until complete dispersion;
(b) adding the fenofibrate and/or the fenofibric acid to the mixture obtained in the preceding step under agitation until complete dispersion; then,
(c) grinding the mixture obtained in the preceding step in the presence of beads at a power sufficient to achieve an average particle size (D50) of the fenofibrate of less than 250 nm;
(d) optionally, homogenizing the ground suspension obtained in the preceding step;
(e) adjusting the quantity of cellulose derivative and, optionally, of water.

This method for preparing the suspension, consisting of adding part of the cellulose derivative before grinding and the remainder after grinding, is advantageous. Indeed, it makes it possible to limit the viscosity of the suspension and to improve the effectiveness of the grinding.

The proportion of cellulose derivative mixed in step (a) advantageously is between 20% and 40%, preferentially roughly 30%, of the quantity of the cellulose derivative in the final suspension.

Step (a) can be broken down into a step of addition of the cellulose derivative and agitation until complete dissolution, a step of addition of the surfactant and agitation until complete dissolution, and, optionally, a step of addition of the phospholipid and agitation until complete dispersion. These steps can occur in any order. Preferentially, the cellulose derivative is added before the surfactant.

Grinding is carried out in the presence of beads. They are preferentially made of polymer, advantageously of polystyrene or zirconium and/or yttrium oxide. Their diameter is between 0.1 mm and 0.8 mm, preferentially between 0.2 mm and 0.6 mm.

The chamber of the mill is filled to at least 70% capacity with beads, preferentially at least 80% capacity. The temperature of the product during grinding is maintained below 35° C., preferentially 30° C. Advantageously, the particle size distribution is measured using laser diffraction or light scattering each hour and grinding is stopped when the diameter decreases no further.

The suspension preparation method of the invention can comprise one or more steps of addition of antifoaming agent, advantageously dimethicone and/or simethicone. These steps can occur before or after grinding. Preferentially, at least one step of addition of antifoaming agent is carried out before grinding to limit or to avoid the formation of foam during grinding.

The present invention also relates to a method for preparing microgranules or granules of fenofibrate and/or fenofibric acid comprising one or more layers of the suspension of the invention, advantageously two layers.

Preferably, the microgranules or granules of the invention comprise fenofibrate or fenofibric acid.

Microgranules are spherical supports on which a suspension of active ingredient can be sprayed. They can be of any chemical nature and preferentially contain sugar and starch, cellulose, or magnesium aluminosilicate.

Granules are particles of more or less irregular shape obtained by the spraying of an active ingredient on an excipient in a more or less fine powder state or by a wet granulation method.

According to a first variant, the microgranules or granules of fenofibrate and/or fenofibric acid of the invention are prepared by one or more assemblies, advantageously two, of the suspension of the invention on neutrals.

Neutral microgranules have a particle size between 200 microns and 1000 microns, preferably between 400 microns and 600 microns.

The assembly is carried out in a sugar-coating pan, in a perforated coating pan or in a fluidized bed, preferably in a fluidized bed.

The assembly on neutral microgranules can be carried out by spraying an aqueous suspension of the invention.

The microgranules of the invention assembled on neutrals are particularly suited to the preparation of capsules.

The microgranules of the invention assembled on neutrals advantageously comprise an overcoating step after the assembly steps, and preferentially a lubrication step after the overcoating step.

The layer of overcoating preferentially consists of a cellulose derivative, advantageously of HPMC. The proportion of overcoating cellulose derivative in the overcoating composition is at least 3%.

The preferred lubricant is talc.

According to a second variant, the granules of fenofibrate and/or fenofibric acid of the invention are prepared by assembly on granules of excipient.

Suitable excipients comprise sugars, preferentially lactose, and microcrystalline cellulose, for example Avicel PH200.

The excipients used are preferentially excipients known to those persons skilled in the art to have good properties of compression, namely excipients used to prepare granules intended to be compressed.

The second variant of the preparation of granules of fenofibrate and/or fenofibric acid of the invention is thus particularly suited to the preparation of granules intended for the preparation of tablets.

Another object of the invention relates to microgranules or granules of fenofibrate and/or fenofibric acid which can be obtained by the method for preparing microgranules or granules of fenofibrate and/or fenofibric acid of the invention.

The microgranules or granules of fenofibrate and/or fenofibric acid of the invention advantageously comprise between 1% and 10%, preferably between 3% and 6%, by weight of phospholipid.

The microgranules or granules of fenofibrate and/or fenofibric acid of the invention advantageously comprise more than 60% by weight of fenofibrate.

The microgranules or granules of fenofibrate and/or fenofibric acid of the invention advantageously comprise between 1% and 10%, preferably between 3% and 5%, by weight of surfactant.

The microgranules or granules of fenofibrate and/or fenofibric acid of the invention advantageously comprise between 7% and 20%, preferably between 10% and 15%, by weight of cellulose derivative, advantageously of hydroxypropyl methylcellulose, as a binder and a solubilization adjuvant.

The microgranules or granules of fenofibrate and/or fenofibric acid of the invention advantageously comprise between 0% and 5%, preferably between 1% and 3%, by weight of lubricant.

The microgranules or granules of fenofibrate and/or fenofibric acid of the invention advantageously comprise between 0% and 7%, preferably between 0% and 3%, by weight of antifoaming agent.

The microgranules of fenofibrate and/or fenofibric acid of the invention advantageously comprise, from the interior toward the exterior:
a core comprised of a neutral,
two coating layers comprised of the suspension of the invention,
a layer of overcoating, advantageously comprising HPMC,
a layer of lubricant, advantageously of talc.

The granules of fenofibrate and/or fenofibric acid advantageously comprise, from the interior toward the exterior:
a core comprised of an excipient, advantageously of directly compressible lactose such as, for example, lactose DCL21,
a coating layer comprised of the suspension of the invention.

The invention also relates to a solid dosage pharmaceutical formulation comprising the microgranules or granules of fenofibrate and/or fenofibric acid of the invention.

Preferably, the solid dosage pharmaceutical formulation of the invention comprises the microgranules or granules of fenofibrate or fenofibric acid of the invention.

The pharmaceutical formulation of the invention advantageously comprises the microgranules or granules of fenofibrate of the invention in a quantity equivalent to a dose of fenofibrate between 20 mg and 200 mg, preferably between 110 mg and 145 mg, in a particularly preferred manner between 110 mg and 120 mg or between 30 mg and 40 mg, for example equal to 115 mg, 120 mg, 130 mg or 145 mg.

The pharmaceutical formulation of the invention comprising the microgranules or granules of fenofibrate of the invention advantageously has the following pharmacokinetic parameters:

(a) AUC∞ between 105000 and 180000 ng h/ml, and

(b) Cmax between 6500 and 12000 ng/ml,

with Cmax signifying the maximum plasma concentration expressed in ng/ml, and AUC∞ signifying the area under the curve from 0 to infinity, expressed in ng h/ml.

The pharmaceutical formulation of the invention advantageously comprises the microgranules or granules of fenofibric acid of the invention in a quantity equivalent to a dose of fenofibric acid between 18 mg and 180 mg, preferably equal to 135 mg.

The pharmaceutical formulation of the invention is provided in the form of capsules, of orodispersible tablets or of tablets.

For the gelatin capsule form, the microgranules of fenofibrate and/or fenofibric acid are prepared according to the first variant. The suspension of the invention is layered on neutrals, in one or more layers, and then the microgranules are coated with an external layer of overcoating before their lubrication.

For the tablet form, the granules of fenofibrate and/or fenofibric acid of the invention are prepared according to the second variant. The suspension of the invention is assembled on granules of excipient and then the granules are mixed with compression adjuvants such as disintegrants, diluents, binders and lubricants.

Among disintegrants, cross-linked carboxymethyl cellulose derivatives, polyvinylpyrrolidone derivatives and starch derivatives are preferred.

Diluents include sugars, preferentially lactose, and cellulose derivatives, notably microcrystalline cellulose.

Among lubricants, magnesium stearate and sodium stearyl fumarate are preferred.

The invention also relates to a pharmaceutical formulation of fenofibrate and/or fenofibric acid of the invention for the treatment of hypertriglyceridemia, hypercholesterolemia or hyperlipidemia.

Advantageously, said treatment has the same pharmacokinetic profile of fenofibrate and/or of fenofibric acid whether the patient has consumed a high-fat meal or has fasted.

The invention also relates to the use of the microgranules or granules of the invention for the manufacture of a drug intended for the treatment of hypertriglyceridemia, hypercholesterolemia or hyperlipidemia.

### FIGURES

FIG. 1 represents the dissolution profile of batches of three assemblies (D05274) and two assemblies (D05291) of the formulation of example 2C.

FIG. 2 represents the dissolution profile of two assemblies as a function of time in minutes, wherein the grinding temperature is less than 30° C. (example 2D)

FIG. 3 represents the dissolution profile of the capsules of example 2E (D05300) versus Antara® and Tricor®.

FIG. 4 shows the diffractograms of fenofibrate (at top), of anhydrous lactose (in the middle) and of granules assembled on anhydrous lactose (at bottom) (example 3).

FIG. 5 shows the diffractograms of granules (at top), and of two batches of tablets (in the middle and at bottom). All of the peaks of the tablets can be attributed to fenofibrate or to anhydrous lactose. They thus do not contain an amorphous phase of fenofibrate (example 3).

FIG. 6 shows changes in the average diameter of the fenofibrate particles measured in nm (left-hand scale) as a function of viscosity (right-hand scale) measured in cP and of time in min (X-axis) (example 1).

FIG. 7 shows the in vivo release profile of a formulation of nanonized fenofibrate in capsule form comprising 0.3% soy lecithin in the suspension (example 4).

FIG. 8 shows the in vivo release profile of 145 mg tablets of the nanonized fenofibrate of the invention (TEST) and that of Lipanthyl® (REFERENCE) in fasting subjects.

FIG. 9 shows the in vivo release profile of 145 mg tablets of the nanonized fenofibrate of the invention (TEST) and that of Lipidil EZ® (REFERENCE) in fasting subjects.

The invention is illustrated in a nonrestrictive way by the following examples.

### EXAMPLE 1

### Preparation of a Suspension of Nanonized Fenofibrate

The formula of the suspension is as follows:

| | | Centesimal |
|---|---|---|
| Batch | Batch mass (g) | formula |
| Fenofibrate | 437.3 | 17.49 |
| HPMC | 82.7 | 3.31 |
| 35% dimethicone | 4.8 | 0.19 |
| 30% simethicone | 0.8 | 0.03 |
| Sodium lauryl | 22.3 | 0.89 |
| sulfate | | |
| Purified water | 1952.2 | 78.08 |
| Total | 2500.1 | 100.00 |

Grinding is carried out in a Netzsch Labstar LS1 wet mill loaded to 88% capacity with 0.3 mm diameter ZrO₂Y₂O₃ beads. The grinding parameters are presented below.

| Beads | 0.3 mm zirconium oxide, |
|---|---|
| | 88% |
| Grinder speed (rpm) | 3000, i.e., 7.9 m/s |
| Product temperature (° C.) | 39 |
| Energy (kWh) | 9.8 |

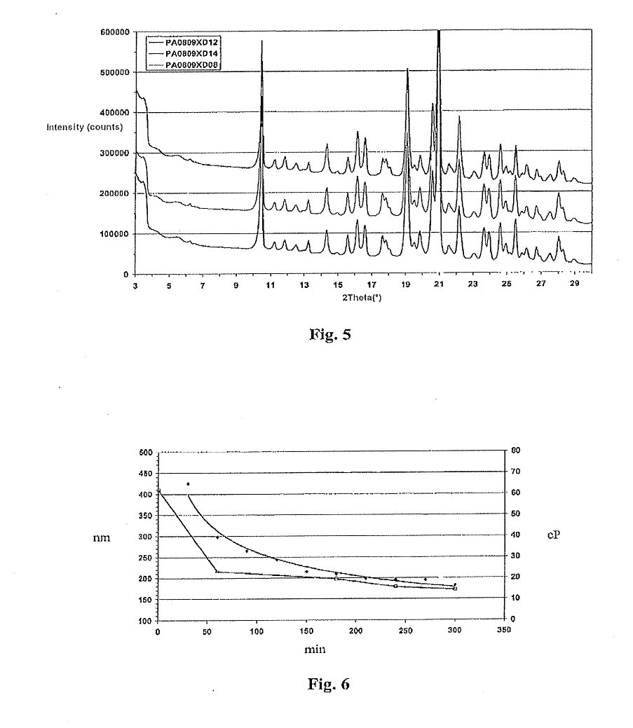

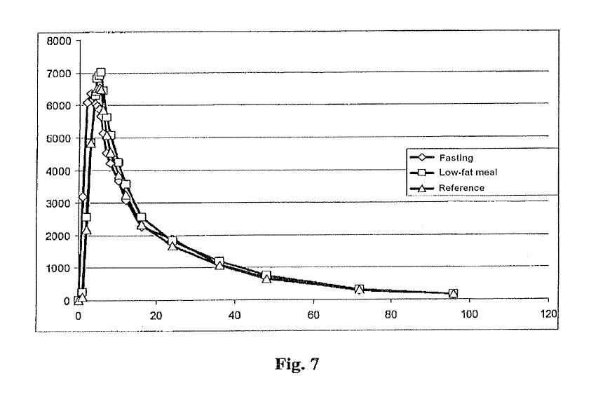

The average diameter of the fenofibrate after 5 h of grinding is 180 nm. The viscosity of the suspension decreases during the process, which is a sign of the alteration of the chains of the HPMC (see FIG. 6).

In this state, the suspension does not have a sufficient binding capacity to be able to be sprayed on the granules to obtain an acceptable content.

Removing part of the HPMC yields a less viscous suspension whose viscosity does not change during milling. The residual HPMC is added after grinding, thus preserving its binding capacity.

### EXAMPLE 2

### Preparation of a Formulation of Nanonized Fenofibrate in Capsule Form

A. Formula of the Suspension

1. Formula

The formula of the assembly suspensions and information on the raw materials used are presented in table 1. Three suspensions are successively milled. Part of the HPMC (30%) is introduced before milling. The remaining 70% is added after milling.

TABLE 1
Formula of assembly suspensions

| | | |
|---|---|---|
| | | CENTESIMAL |
| | FORMULA | FORMULA |
| MATERIAL | (g) | (%) |

BEFORE MILLING

| | | |
|---|---|---|
| Purified water | 1376.60 | 74.49 |
| 30% simethicone emulsion | 0.80 | 0.04 |
| 35% dimethicone emulsion | 4.70 | 0.25 |
| Sodium lauryl sulfate | 22.00 | 1.19 |
| HPMC 603 | 24.00 | 1.30 |
| Fenofibrate | 420.00 | 22.73 |
| Total | 1848.10 | 100.00 |

AFTER MILLING

| | | | |
|---|---|---|---|
| Milled | Purified water | 1288.63 | 76.89 |
| suspension | 30% simethicone | 0.75 | |
| | emulsion | | |
| | 35% dimethicone | 4.40 | |
| | emulsion | | |
| | Sodium lauryl sulfate | 20.59 | |
| | HPMC 603 | 22.47 | |
| | Fenofibrate | 393.16 | |
| | Milled suspension | 1730.00 | |
| | total | | |

| | | |
|---|---|---|
| Purified water | 468.00 | 20.80 |
| HPMC 603 | 52.00 | 2.31 |
| Total | 2250.00 | 100.00 |
| Quantity used for the assembly | 2216.00 | 98.49 |

2. Particle Size Measurements

The suspensions of fenofibrate were analyzed by measuring particle size after milling.

The results are presented in table 2.

TABLE 2
Particle size measurements

| | | | |
|---|---|---|---|
| DETERMINATION | METHODS | SPECIFICATIONS | RESULTS |

Particle size measurements

| | | | |
|---|---|---|---|
| Milled | Coulter N4 + | <300 nm | 279 nm |
| suspension | unimodal | | |
| no. 1 | mode | | |
| Milled | | <300 nm | 293 nm |
| suspension | | | |
| no. 2 | | | |
| Milled | | <300 nm | 298 nm |
| suspension | | | |
| no. 3 | | | |

B. Preparation of the Suspension

Grinding of the fenofibrate suspension is carried out with a Netzsch Labstar LS1 mill equipped with 0.2 mm diameter ZrO₂Y₂O₃ beads.

The useful volume of the grinding chamber is 0.57 liters. The grinding chamber was filled to 90% capacity with the beads.

The principal grinding parameters are summarized in table 3.

TABLE 3
Grinding parameters

| | GRINDING | GRINDING | GRINDING |
|---|---|---|---|
| | NO. 1 | NO. 2 | NO. 3 |
| GRINDER SPEED | 2200-2400 | 2200-2500 | 2200-2300 |
| (rpm) | | | |
| AVERAGE | 279 | 293 | 298 |
| DIAMETER AFTER | | | |
| GRINDING (nm) | | | |

C. Preparation of Microgranules Undergoing Two Versus Three Grindings-Assemblies

The suspension of step A is assembled on neutrals.

Two or three successive grindings-assemblies are performed to obtain the desired content. The microgranules obtained are lubricated with talc.

The theoretical formula of the granules is indicated in table 4.

TABLE 4
Theoretical formula of the fenofibrate granules

| | BATCH OF 3 | | BATCH OF 2 | |
|---|---|---|---|---|
| | ASSEMBLIES | | ASSEMBLIES | |
| | QUAN- | CENTESIMAL | QUAN- | CENTESIMAL |
| | TITY | FORMULA | TITY | FORMULA |
| MATERIAL | (g) | (%) | (g) | (%) |
| Neutrals 30 | 300.00 | 23.59 | 300.00 | 38.14 |
| 30% | 0.44 | 0.03 | 0.22 | 0.03 |
| simethicone | | | | |
| emulsion | | | | |
| 35% | 3.03 | 0.24 | 1.52 | 0.19 |
| dimethicone | | | | |
| emulsion | | | | |
| Sodium | 40.57 | 3.19 | 20.28 | 2.58 |
| lauryl | | | | |
| sulfate | | | | |
| HPMC 603 | 146.68 | 11.54 | 73.34 | 9.33 |
| Fenofibrate | 774.44 | 60.91 | 387.22 | 49.23 |
| Talc | 6.33 | 0.50 | 3.91 | 0.50 |
| Total | 1271.49 | 100.00 | 786.49 | 100.00 |

The assembly of the fenofibrate suspension is carried out in a GPCG1 fluidized bed.

Lubrication is carried out in a conventional coating pan with 0.5% talc relative to the mass of the granules produced.

The principal parameters of assembly and drying are summarized in table 5.

TABLE 5
Parameters of assembly-drying

| | | PRODUCT | ATOMIZA- | |
|---|---|---|---|---|
| | SPRAYING | TEMPERA- | TION | AIR FLOW |
| | RATE | TURE | PRESSURE | RATE |
| | (g/rain) | (° C.) | (bar) | (m/s) |
| Assembly 1 | 2.4-5.5 | 40-44 | 1.0 | 7 |
| Drying 1 | / | 41 | / | 5 |
| Assembly 2 | 4.7-5.3 | 39-43 | 1.0 | 7 |
| Drying 2 | / | 41 | / | 7 |
| Assembly 3 | 4.6-5.7 | 39-43 | 1.0 | 7 |
| Drying 3 | / | 42 | / | 7 |

The principal analytical results are summarized in table 6.

TABLE 6
Masses, yields, titers and dissolution profiles of the granules

| | | |
|---|---|---|
| BATCH | 3 ASSEMBLIES | 2 ASSEMBLIES |

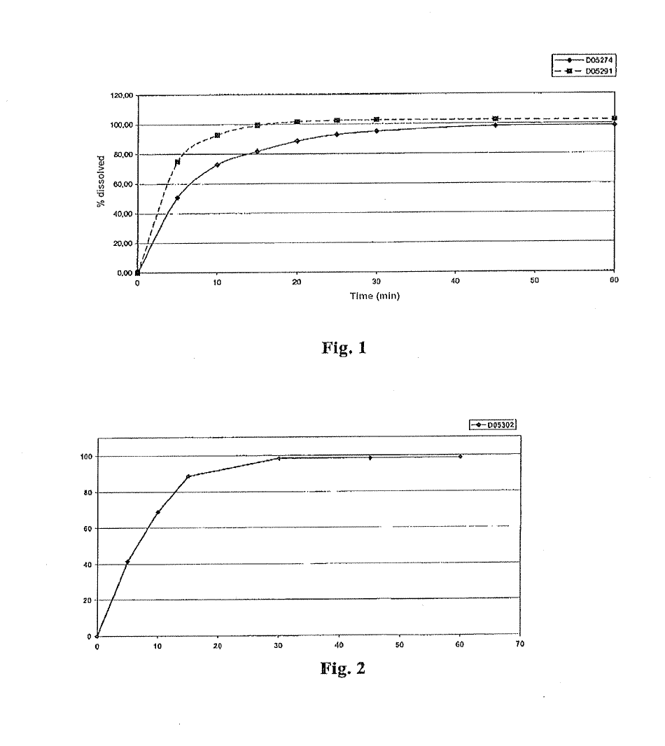

MICROGRANULES

| | | |
|---|---|---|
| MASS YIELD | 93.0% | 91.7% |
| THEORETICAL TITER | 609.08 | 492.34 |
| (mg/g) | | |
| Dissolution (see | 5 min: 50.7% | 5 min: 75.1% |
| FIG. 1) | 10 min: 72.8% | 10 min: 92.7% |
| | 15 min: 81.7% | 15 min: 99.2% |
| | 30 min: 95.1% | 30 min: 102.7% |
| | 45 min: 98.4% | 45 min: 102.7% |
| | 60 min: 98.5% | 60 min: 102.4% |

It can be observed in FIG. 1 that the dissolution profile is faster for the granules with two layers than for the granules with three layers.

D. Preparation of Microgranules Undergoing Two Versus Four Grindings-Assemblies

The grinding suspensions are prepared in the same way as before: the beads used are 0.2 mm in diameter. The temperature during grinding is maintained below 30° C.

The theoretical formula of the granules is indicated in table 7.

TABLE 7
Theoretical formula of the fenofibrate granules

| | BATCH OF 4 | | BATCH OF 2 | |
|---|---|---|---|---|
| | ASSEMBLIES | | ASSEMBLIES | |
| | QUAN- | CENTESIMAL | QUAN- | CENTESIMAL |
| | TITY | FORMULA | TITY | FORMULA |
| MATERIAL | (g) | (%) | (g) | (%) |
| Neutrals 30 | 300.00 | 17.08 | 300.00 | 38.14 |
| 30% | 0.66 | 0.04 | 0.22 | 0.03 |
| simethicone | | | | |
| emulsion | | | | |
| 35% | 4.55 | 0.26 | 1.52 | 0.19 |
| dimethicone | | | | |
| emulsion | | | | |
| Sodium | 60.85 | 3.46 | 20.28 | 2.58 |
| lauryl | | | | |
| sulfate | | | | |
| HPMC 603 | 220.02 | 12.53 | 73.34 | 9.33 |
| Fenofibrate | 1161.66 | 66.14 | 387.22 | 49.23 |
| Talc | 8.74 | 0.50 | 3.91 | 0.50 |
| Total | 1756.48 | 100.00 | 786.49 | 100.00 |

| | | |
|---|---|---|
| Theoretical | 661.35 | 492.34 |
| titer | | |
| (mg/g) | | |

The assembly of the suspension of nanonized fenofibrate is carried out in a GPCG1 fluidized bed.

Lubrication is carried out in a conventional coating pan with 0.5% talc relative to the mass of the granules produced.

The principal parameters of assembly and drying are summarized in table 8.

TABLE 8
Parameters of assembly-drying

| | | PRODUCT | ATOMIZA- | |
|---|---|---|---|---|
| | SPRAYING | TEMPERA- | TION | AIR FLOW |
| | RATE | TURE | PRESSURE | RATE |
| | (g/min) | (° C.) | (bar) | (m/s) |
| Assembly 1 | 2.4-5.6 | 40-44 | 1.0 | 7-8 |
| Drying 1 | / | 40 | / | 5 |
| Assembly 2 | 5.1-5.5 | 40-42 | 1.0 | 7-7.5 |
| Drying 2 | / | 40 | / | 7 |
| Assembly 3 | 4.8-6.2 | 40-42 | 1.0 | 7-7.5 |
| Drying 3 | / | 41 | / | 7 |
| Assembly 4 | 5.3-6.0 | 39-41 | 1.0 | 7-7.5 |
| Drying 4 | / | 41 | / | 7 |

The characteristics of the granules obtained are presented in table 9.

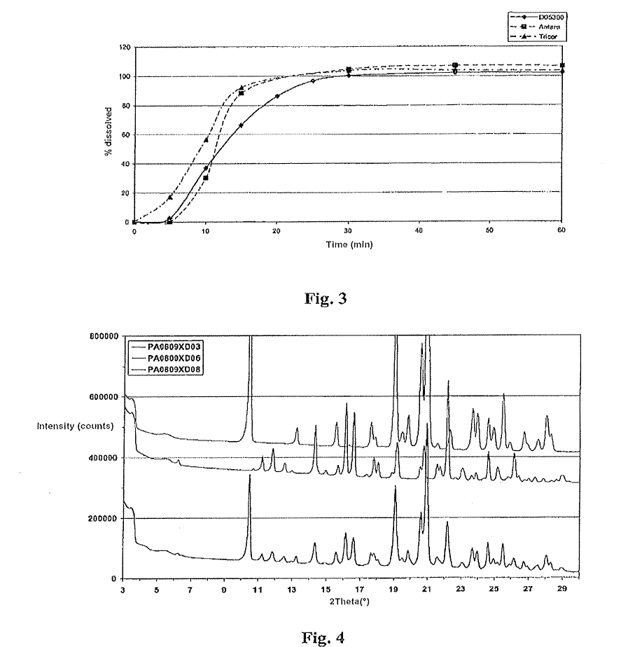

TABLE 9
Characteristics of the granules

| BATCH | 4 ASSEMBLIES | 2 ASSEMBLIES |
|---|---|---|
| MASS YIELD | 90.9% | 91.8% |
| THEORETICAL TITER | 661.35 | 492.34 |
| (mg/g) | | |
| Dissolution (see | N/A | 5 min: 41.5% |
| FIG. 2) | | 10 min: 68.9% |
| | | 15 min: 88.6% |
| | | 30 min: 98.4% |
| | | 45 min: 98.4% |
| | | 60 min: 98.6% |

The two-assembly microgranules are put into #2 capsules (theoretical fenofibrate content: 130 mg).

E. Preparation of the Capsules

The two-assembly granules are put into capsules in a content of 130 mg. The content of fenofibrate granules is 475.9 mg/g. The filling mass is thus 273.2 mg of granules per gelatin capsule.

The average mass of the full gelatin capsules is 337.1 mg.

TABLE 10
Analytical results for the capsules

| TEST | RESULTS |
|---|---|
| Content uniformity | 102.2% |
| | 102.1% |
| | 101.3% |
| | 100.4% |
| | 100.3% |
| | 104.0% |
| | 100.4% |
| | 102.7% |
| | 101.6% |
| | 104.9% |
| | X = 102.0%/SD = 1.5/VA = 4.2 |
| | Conforms: level 1 |
| Dissolution (see FIG. 3) | 5 min: 2.7% |
| | 10 min: 37.0% |
| | 15 min: 66.4% |
| | 20 min: 86.2% |
| | 25 min: 96.8% |
| | 30 min: 100.2% |
| | 45 min: 102.0% |
| | 60 min: 102.1% |
| Fenofibrate content | 132.6 mg/gelatin capsule |

### EXAMPLE 3

### Preparation of a Formulation of Nanonized Fenofibrate in Tablet Form

A. Preparation of the Suspension
a. The simethicone is added to the water under agitation.
b. The dimethicone is added and maintained under agitation until complete dissolution.
c. The sodium lauryl sulfate is added and maintained under agitation until complete dissolution.
d. The HPMC 603 is added and maintained under agitation until complete dissolution.
e. The fenofibrate is added and maintained under agitation until complete dispersion.
f. The agitation is maintained until complete dispersion.

This suspension is ground in a wet mill loaded (80%) with 0.2 mm zirconium oxide beads. Grinding is carried out at low speed (roughly 1500 rpm to 2500 rpm). The temperature of the product is maintained below 35° C.

B. Formula of the Suspension

TABLE 11
Formulation of the suspension

| | MASS (g) | PERCENTAGE |
|---|---|---|
| Fenofibrate | 387.5 | 22.73 |
| HPMC | 22.2 | 1.30 |
| 35% dimethicone | 4.3 | 0.25 |
| 30% simethicone | 0.7 | 0.04 |
| Sodium lauryl | 19.8 | 1.16 |
| sulfate | | |
| Water | 1270.1 | 74.51 |
| Total | 1704.6 | 100.00 |

C. Preparation of the Granules

The suspension is layered on granules of sugar (400-600 μm) in a FBC (GPCG1, Glatt, bottom spray, 1.2 mm), on granules of sucrose (540 μm diameter), on granules of microcrystalline cellulose (Ethispheres 100, Cellets 100, Avicel PH200), or on directly compressible lactose.

Granules of lactose DLC21 are selected for the manufacture of tablets because of their desirable compression and dissolution properties.

TABLE 12
Formula of granules on Avicel PH200

| | MASS (g) | PERCENTAGE (%) |
|---|---|---|
| Lactose DCL21 | 700.00 | 62.83 |
| Fenofibrate | 326.01 | 29.26 |
| Simethicone (dry) | 0.18 | 0.02 |
| Dimethicone (dry) | 1.27 | 0.11 |
| HPMC 603 | 61.62 | 5.53 |
| Sodium lauryl | 17.04 | 1.53 |
| sulfate | | |
| Talc | 8.00 | 0.72 |
| Water | 1457.08* | /* |
| Total dry mass | 1114.12 | 100.00 |

*removed during the drying step

D. Preparation of the Tablets

TABLE 13
Formulation of 145 mg fenofibrate tablets

| | FORMULA PER UNIT | |
|---|---|---|
| | (mg) | PERCENTAGE (%) |
| Lactose DCL21 | 449.8 | 69.14 |
| Fenofibrate | 144.9 | 22.27 |
| Simethicone (dry) | 0.1 | 0.02 |
| Dimethicone (dry) | 0.6 | 0.09 |
| HPMC 603 | 27.4 | 4.21 |
| Sodium lauryl | 7.6 | 1.17 |
| sulfate | | |
| Ac-Di-Sol | 19.6 | 3.01 |
| Magnesium | 0.6 | 0.09 |
| stearate | | |
| Total | 650.6 | 100.00 |

The granules containing lactose are compressed with lactose as a diluent, croscarmellose sodium as a disintegrant and magnesium stearate as a lubricant.

The compression parameters are as follows:
SVIAC PR12 compressor,
15 mm×7 mm oblong punches,
average mass: 654.1 mg/tablet,
average hardness: 74.4 N.

E. X-Ray Diffraction Crystallography Analysis

FIG. 4 shows the diffractograms of the fenofibrate (at top), of the anhydrous lactose (in the middle) and of the granules assembled on anhydrous lactose (at bottom). All of the peaks of the granules can be attributed to the fenofibrate or to the anhydrous lactose. The grinding and assembly steps do not induce the appearance of amorphous fenofibrate.

FIG. 5 shows the diffractograms of the granules (at top), and of two batches of tablets (in the middle and at bottom). All of the peaks can be attributed to the fenofibrate or to the anhydrous lactose. Thus, they do not contain fenofibrate in an amorphous phase.

F. Pharmacokinetic Analyses

1—Comparison of the in vivo release profile of the tablets containing granules with 145 mg of nanonized fenofibrate in fasting subjects (TEST) with that of tablets marketed under the name Lipanthyl® (granules with 145 mg of nanonized fenofibrate) in fasting subjects.

Lipanthyl® is regarded as the reference product.

This study is carried out with 31 subjects. Blood samples are taken at regular time intervals and fenofibric acid is assayed.

The results are presented in tables 19 and 20.

The following abbreviations are used in the present application:
Cmax: maximum plasma concentration expressed in ng/ml
Tmax: time necessary to reach Cmax
AUCt: area under the curve from 0 to t, expressed in ng h/ml
t represents last time with a measurable concentration
AUC∞: area under the curve from 0 to infinity, expressed in ng h/ml

These results show that the composition administered under the “TEST” conditions is bioequivalent to the reference product Lipanthyl® administered to a fasting subject.

TABLE 19

| | TEST | | REFERENCE | |
|---|---|---|---|---|
| PARAMETER | MEAN | CV (%) | MEAN | CV (%) |
| Cmax (ng/ml) | 9416.8 | 20.3 | 10385.4 | 88.8 |
| tmax (hours) | 2.00 | 49.5 | 2.00 | 40.9 |
| AUCt (ng · h/ml) | 158561.9 | 53.6 | 158012.6 | 39.2 |
| AUCα (ng · h/ml) | 169277.6 | 66.1 | 163551.1 | 41.0 |
| AUCt/α (%) | 96.46 | 5.0 | 97.19 | 2.2 |

TABLE 20

| | TEST | | REFERENCE | |
|---|---|---|---|---|
| PARAMETER | MEAN | CV (%) | MEAN | CV (%) |
| Cmax (ng/ml) | 9416.8 | 20.3 | 10385.4 | 18.8 |
| tmax (hours) | 2.00 | 49.5 | 2.00 | 40.9 |
| AUCt (ng · h/ml) | 158561.9 | 53.6 | 158012.6 | 39.2 |
| AUCα (ng · h/ml) | 169277.6 | 66.1 | 163551.1 | 41.0 |
| AUCt/α (%) | 96.46 | 5.0 | 97.19 | 2.2 |

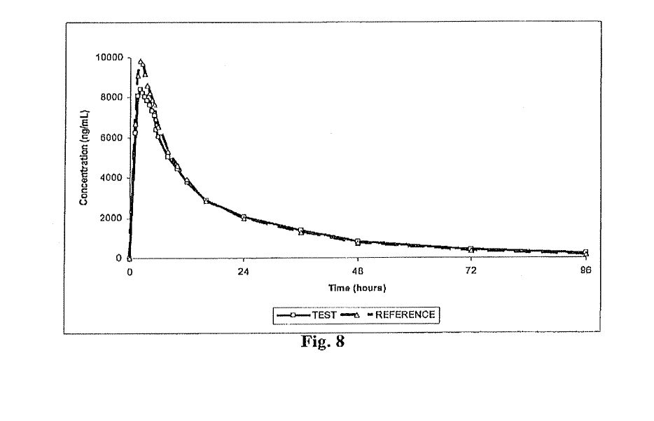

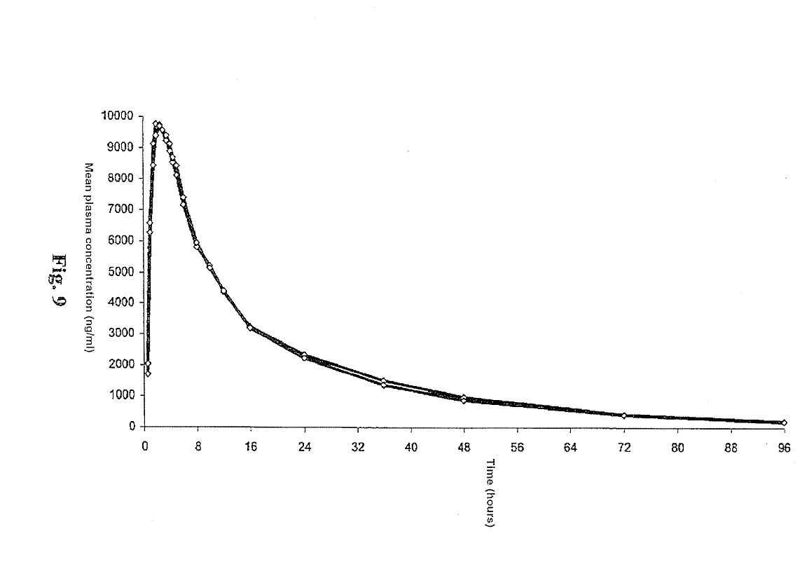

The results are represented graphically in FIG. 8.

2—Comparison of the in vivo release profile of tablets containing granules with 145 mg of nanonized fenofibrate in fasting subjects (TEST) with that of tablets marketed under the name Lipidil EZ® (granules with 145 mg of nanonized fenofibrate) in fasting subjects.

Lipidil EZ® is regarded as the reference product.

This study is carried out with 19 subjects. Blood samples are taken at regular time intervals and fenofibric acid is assayed.

The results are presented in tables 21 and 22.

TABLE 21

| PARAMETER | TEST | | REFERENCE | |
|---|---|---|---|---|
| Cmax (ng/ml) | 10615.4 | (26.2) | 10920.6 | (21.3) |
| tmax (hours) | 2.00 | [1-3.53] | 2.00 | [1.00-5.00] |
| AUCt* (ng · h/ml) | 173297.4 | (38.2) | 167147.5 | (35.1) |
| AUCα* (ng · h/ml) | 179028.2 | (38.5) | 171952.5 | (35.8) |
| AUCt/α* (%) | 96.92 | (2.1) | 97.39 | (1.5) |
| t½el* (h) | 19.09 | (26.08) | 18.18 | (23.64) |

TABLE 22

| Parameter | Ratio (%) | IC90% |
|---|---|---|
| Cmax | 96.13 | X |
| AUCt* | 102.94 | [98.61-107.46] |
| AUCα* | 103.41 | [99.25-107.75] |

These results show that the composition administered under the “TEST” conditions is bioequivalent to the reference product Lipidil EZ® administered to a fasting subject.

The results are represented graphically in FIG. 9.

### EXAMPLE 4

### Preparation of a Formulation of Nanonized Fenofibrate in the Form of a Capsule Comprising 0.3% Soy Lecithin in the Suspension

A. Preparation of the Suspension

Preparation of the Suspension to be Milled:
introduce water into a container of suitable size,
add the simethicone suspension under constant agitation,
add the HPMC 603 under agitation,
add the sodium lauryl sulfate under constant agitation,
continue the agitation until perfect homogenization,
add the soy lecithin under constant agitation,
continue the agitation until perfect homogenization,
add the fenofibrate under constant agitation,
continue the agitation for least 30 minutes before transfer of the suspension in the mill, and maintain agitation during grinding.

The chamber (1, 1 l) of the HOSOKAWA 90AHM mill is loaded, to 80% capacity with 0.36-0.5 mm diameter polystyrene beads. The mill is used in recirculation mode. The grinding power is adjusted to achieve the desired particle size (D50<250 nm).

The system is cooled continuously so that the product temperature does not exceed 45° C.

Grinding efficiency is verified by measurement of the particle size (Malvern Mastersizer 2000HydroS).

The grinding parameters are as follows:
Pump speed: 100 rpm
Grinding speed: 2500 rpm
Total grinding time: 300 min

Table 14 presents the quantitative (g) and centesimal (%) formula of the ready-to-spray suspension.

TABLE 14
Quantities of materials per grinding suspension

| | QUANTITATIVE | CENTESIMAL |
|---|---|---|
| Material | FORMULA (g) | FORMULA (%) |
| Purified water | 3652.30 | 78.01 |
| Fenofibrate | 817.11 | 17.45 |
| HPMC 603 | 154.67 | 3.30 |
| Simethicone (30% | 1.54 | 0.03 |
| emulsion) | | |
| Sodium lauryl | 42.76 | 0.91 |
| sulfate | | |
| Soy lecithin | 13.62 | 0.29 |
| Total | 4682.00 | 100.00 |

B. Preparation of the Microgranules

Table 15 presents the centesimal and quantitative formula of the fenofibrate granules.

TABLE 15
Formula of the fenofibrate granules

| | Quantitative | Centesimal |
|---|---|---|
| Material | formula (g) | formula (%) |
| Neutrals 30 | 290.29 | 16.33 |
| Fenofibrate | 1119.84 | 62.99 |
| Soy lecithin | 18.66 | 1.05 |
| 30% simethicone | 0.63 | 0.04 |
| emulsion (dry | | |
| matter) | | |
| HPMC 603 | 211.97 | 11.92 |
| Sodium lauryl | 58.61 | 3.30 |
| sulfate | | |
| Talc | 31.79 | 1.79 |
| HPMC 606 | 45.90 | 2.58 |
| Purified water | — | —* |
| Total | 1777.69 | 100.00 |

*solvent evaporated during the process

The manufacturing process consists of:
(1) Grinding the fenofibrate in suspension in water+simethicone+sodium lauryl sulfate+soy lecithin+HPMC 603
(2) Spraying on a neutral support
(3) Overcoating with HPMC 606/talc

The assembly is carried out in a fluidized bed to obtain a fenofibrate content of the microgranules greater than 600 mg/g.

The assembly parameters are as follows:

| Steps | Parameters |
|---|---|
| Assembly | Inflow air temperature: 50-60° C. |
| | Product temperature: 35-45° C. |
| | Atomization pressure: 1-1.5 bar |
| | Air flow rate: 4-8 m/s |
| Drying | Inflow air temperature: 50° C. |
| | Product temperature: 40° C. |
| | Drying time: 10 min |

Approximately 1700 g of assembled granules are recovered. They are overcoated with HPMC 606/talc.

The quantitative and centesimal formula of the coating suspension is as follows:

The coating parameters are as follows:

| Steps | Parameters |
|---|---|
| Assembly | Inflow air temperature: 50-60° C. |
| | Product temperature: 35-45° C. |
| | Atomization pressure: 1-1.5 bar |
| | Air flow rate: 4-8 m/s |
| Drying | Inflow air temperature: 50° C. |
| | Product temperature: 40° C. |
| | Drying time: 10 min |

The assembled and overcoated granules are lubricated with 0.5% talc relative to the total mass.

C. Preparation of the Gelatin Capsules

The granules of the step B are put into gelatin capsules in a content of 130 mg.

### EXAMPLE 5

### Pharmacokinetic Studies of the Formulation of Example 4C

A. Comparison of the In Vivo Release Profile of Capsules Containing Granules with 130 Mg of Nanonized Fenofibrate and Comprising Soy Lecithin in Fasting Subjects (Test 1) to that of Capsules Marketed Under the Name Antara® (Granules with 130 Mg of Micronized Fenofibrate) in Subjects Having Just Consumed a Low-Fat Meal.

Antara® is regarded as the reference product.

This study is carried out with 12 subjects. Blood samples are taken at regular time intervals and fenofibric acid is assayed.

The results are presented in table 16.

TABLE 16

| | | | | 90% confidence | |
|---|---|---|---|---|---|
| Parameters | Test1 | Reference | Ratio | interval | |
| Cmax | 6784.9 | 6767.4 | 100.26 | 91.18 | 110.24 |
| AUCt | 122904.6 | 118176.1 | 104.00 | 98.32 | 110.01 |
| AUC∞ | 127668.3 | 123349.7 | 103.50 | 97.96 | 109.36 |

The following abbreviations are used in the present application:
Cmax: maximum plasma concentration expressed in ng/ml
Tmax: time necessary to reach Cmax
AUCt: area under the curve from 0 to t, expressed in ng h/ml
t represents last time with a measurable concentration
AUC∞: area under the curve from 0 to infinity, expressed in ng h/ml

These results show that the composition administered under the “Test1” conditions is bioequivalent to the reference product Antara® administered in subjects having just consumed a low-fat meal. The reduction in the size of the fenofibrate particles combined with the addition of soy lecithin improved the bioavailability of the product.

B. Comparison of the In Vivo Release Profile of Gelatin Capsules Containing the Granules with 130 Mg of Nanonized Fenofibrate and Comprising Soy Lecithin in Subjects Having Just Consumed a Low-Fat Meal (Test 2) with that of Gelatin Capsules Marketed Under the Name Antara® (Granules with 130 Mg of Micronized Fenofibrate) in Subjects Having Just Consumed a Low-Fat Meal.

This study is carried out with 11 and 12 subjects, respectively. Blood samples are taken at regular time intervals and fenofibric acid is assayed.

The results are presented in table 17.

TABLE 17

| | | | | 90% confidence | |
|---|---|---|---|---|---|
| Parameters | Test2 | Reference | Ratio | interval | |
| Cmax | 7333.8 | 6767.4 | 108.37 | 98.25 | 119.53 |
| AUCt | 123093.9 | 118176.1 | 104.16 | 98.27 | 110.41 |
| AUC∞ | 127435.2 | 123349.7 | 103.31 | 97.59 | 109.38 |

These results show that the composition administered under the “Test2” conditions is bioequivalent to the reference product Antara® administered under the same conditions.

C. Comparison of the In Vivo Release Profile of Gelatin Capsules Containing Granules with 130 Mg of Nanonized Fenofibrate and Comprising Soy Lecithin in Subjects Having Just Consumed a Low-Fat Meal (Test 2) with that of Gelatin Capsules Containing Granules with 130 Mg of Nanonized Fenofibrate and Comprising Soy Lecithin in Fasting Subjects (Test 1).

The results are presented in table 18 and FIG. 7.

TABLE 18

| | | | | 90% confidence | |
|---|---|---|---|---|---|
| Parameters | Test1 | Test2 | Ratio | interval | |
| Cmax | 6784.9 | 7333.8 | 92.52 | 83.88 | 102.04 |
| AUCt | 122904.6 | 123093.9 | 99.85 | 94.20 | 105.83 |
| AUC∞ | 127668.3 | 127435.2 | 100.18 | 94.63 | 106.06 |

These results show that the composition of gelatin capsules comprising granules of nanonized fenofibrate and soy lecithin is bioequivalent under the two conditions of administration, i.e., fasting and with a low-fat meal.

1. An aqueous suspension containing fenofibrate and/or fenofibric acid in suspension, a cellulose derivative as a solubilization adjuvant and a surfactant, wherein the fenofibrate and/or the fenofibric acid are in crystalline form, with a melting point in DSC of 79° C. to 82° C. or 179° C. to 183° C., respectively, and wherein the average size of the particles (D50) of fenofibrate or of fenofibric acid is less than 250 nm, preferably 180 nm.

2. A method for preparing a suspension of claim 1, comprising the following steps:
(a) mixing part of the proportion of cellulose derivative in the final suspension, the surfactant and, optionally, the phospholipid with water under agitation until complete dispersion;
(b) adding the fenofibrate and/or the fenofibric acid to the mixture obtained in the preceding step under agitation until complete dispersion; then,
(c) grinding the mixture obtained in the preceding step in the presence of beads at a power sufficient to achieve an average particle size (D50) of the fenofibrate of less than 250 nm;
(d) optionally, homogenizing the ground suspension obtained in the preceding step; and
(e) adjusting the quantity of cellulose derivative and, optionally, of water.

3. The method of claim 2, wherein the proportion of cellulose derivative mixed in step (a) is approximately 30% of the quantity of cellulose derivative in the final suspension.

4. The method of claim 2, wherein the grinding is carried out in the presence of beads of polymer or of zirconium and/or yttrium oxide.

5. The method of claim 2, comprising a step of the addition of antifoaming agent.

6. A method for preparing microgranules or granules of fenofibrate and/or fenofibric acid, comprising a step (a) of the spraying of one or more layers, advantageously two, of a suspension of claim 1 on neutrals or on granules of excipient.

7. The method of claim 6, characterized by a step of overcoating of the coated neutrals obtained in step (a) by a composition comprising at least 3% of a cellulose derivative, preferentially of HPMC.

8. The method of claim 7, characterized by a final step of lubrication, preferentially by talc.

9. The microgranules or granules of fenofibrate and/or fenofibric acid which can be obtained by the method of claim 6.

10. The microgranules or granules of fenofibrate and/or fenofibric acid of claim 9, wherein the proportion of the phospholipid is between 1% and 10%, preferably between 3% and 6%, by weight of the microgranules.

11. The microgranules or granules of fenofibrate and/or fenofibric acid of claim 9, wherein the proportion of fenofibrate is greater than 60% by weight of the microgranules.

12. The microgranules or granules of fenofibrate and/or fenofibric acid of claim 9, wherein the proportion of surfactant is between 1% and 10%, preferably between 3% and 5%, by weight of the microgranules.

13. The microgranules or granules of fenofibrate and/or fenofibric acid of claim 9, wherein the proportion of hydroxypropyl methylcellulose as a binder and a solubilization adjuvant is between 7% and 20%, preferably between 10% and 15%, by weight of the microgranules.

14. The microgranules of fenofibrate and/or fenofibric acid of claim 9, comprising from the interior toward the exterior:
(a) a core comprised of a neutral,
(b) two coating layers comprised of the suspension of claim 1,
(c) a layer of overcoating, advantageously comprising HPMC, and
(d) a layer of lubricant, advantageously of talc.

15. The granules of fenofibrate and/or fenofibric acid of claim 9, comprising from the interior toward the exterior:
(a) a core comprised of an excipient, advantageously of lactose, and
(b) a layer of coating comprised of the suspension of claim 1.

16. A solid dosage pharmaceutical formulation comprising the microgranules or granules of claim 9, comprising between 20 mg and 200 mg of fenofibrate, preferably between 110 mg and 145 mg of fenofibrate.

17. The solid dosage pharmaceutical formulation comprising the microgranules or granules of claim 9, comprising between 18 mg and 180 mg of fenofibric acid.

18. The pharmaceutical formulation of claim 16, comprising between 110 mg and 120 mg of fenofibrate, or between 30 mg and 40 mg of fenofibrate.

19. The pharmaceutical formulation of claim 16, with the following pharmacokinetic parameters:
(a) AUCt between 105000 ng·h/ml and 180000 ng·h/ml, and
(b) Cmax between 6500 ng/ml and 12000 ng/ml.

20. The pharmaceutical formulation of fenofibrate and/or fenofibric acid of claim 18, wherein the pharmacokinetic profile of the fenofibrate is equivalent whether the patient has consumed a high-fat meal or has fasted.
